(19) United States
(12) Patent Application Publication
(10) Pub. No.: US 2004/0059308 A1
(43) Pub. Date: Mar. 25, 2004

(54) Male, post-void, urinary drip napkin and method

(76) Inventor: Ib R. Odderson, Kirkland, WA (US)

Correspondence Address: Dean A. Craine, P.S., Suite 140, 400 - 12th Ave. NE., Bellevue, WA, 98004, US

(21) Appl. No.: 10/254,214

(22) Filed: Sep. 24, 2002

(51) Int. Cl. A61F 13/15; A61F 13/20

(52) U.S. Cl. 604/358

(57) ABSTRACT

A male, post-void, urinary drip napkin designed to be longitudinally aligned and placed over the penis so that the penis may be shaken with one hand without the fingers directly touching the penis or urine. The napkin is made of lightweight, absorbent material capable of being discarded into a toilet or urinal after being used. The napkin includes an open end and an opposite closed end with only a longitudinally aligned, central tab formed on the closed end. The napkin is designed so it can be either folded into a flat, conical configuration that can be slidingly dispensed from a dispenser or stored in its expanded conical shape in the dispenser. During use, the napkin is expanded into a cylindrical configuration and placed over the penis. The top edge of the open end is concave so that the penis may be viewed when the penis is inserted into the napkin.

### BACKGROUND OF THE INVENTION

1. Field of the Invention

This invention relates to sanitary pads, and, more particularly, to disposable sanitary napkins for treating post-void, urinary drip in men.

2. Description of the Related Art

As the baby boomer generation gets older, a greater need arises for a disposable sanitary napkin for men to protect their hands and clothing from post-void, urinary drip. Ideally, the disposable sanitary napkin should be placed around the tip of the penis to allow the penis, surrounded with the napkin, to be shaken, and then the napkin may be flushed down the toilet after use. The napkin should also be designed to be dispensed from a small compact dispenser located near the toilet or urinal.

### SUMMARY OF THE INVENTION

It is an object of the present invention to provide a male, post-void, urinary drip napkin.

It is another object of the present invention to provide such a napkin that can be placed over the tip of the penis and then used to shake the penis to dislodge urine from the urethra.

It is a further object of the invention to provide such a napkin that can be dispensed from a conveniently located dispenser and then flushed down a urinal or toilet after use.

It is another object of the present invention to provide a method for dislodging urine from a penis.

These and other objects are met by a male, post-void, urinary drip napkin designed to be placed over the tip of the penis to capture drops of urine. The napkin is relatively small and made of a lightweight, absorbent material capable of being discarded down a urinal or toilet after use without risk of obstruction. The napkin is either dispensed in a flat, folded, triangular configuration that easily expands into an open-ended, conical-shaped configuration, or dispensed in an open-ended conical configuration. When disposed in an open-ended, conical-shaped configuration, the napkin may be placed longitudinally over the penis. A single, short tab member is formed on the closed end of the napkin which enables the napkin to be easily dispensed from the dispenser, placed over the penis, using one hand. The short tab also allows the penis to be shaken without the fingers directly touching the penis. In the preferred embodiment, the napkin is a continuous structure with no side seams so that the bulk of the napkin is relatively small and easily dissolves in water for easy disposal in a urinal.

### BRIEF DESCRIPTION OF THE DRAWINGS

FIG. 1 is a front plan view of a plurality of longitudinally aligned and stacked male, post-void, urinary drip napkins disclosed herein.

FIG. 2 is a bottom elevational view of the napkin positioned in a flat orientation.

FIG. 3 is a bottom elevational view of the napkin shown in FIG. 2, shown expanded into a conical configuration placed around a penis.

FIG. 4 is an illustration showing the napkin disposed around a penis and being held by the tab and shaken to remove post-void, urinary drips.

FIG. 5 is a perspective view of a plurality of napkins shown in FIG. 1 longitudinally inside a dispenser.

FIG. 6 is a perspective view of a plurality of napkins aligned longitudinally and rolled into a second embodiment of the dispenser.

### DESCRIPTION OF THE PREFERRED EMBODIMENT(S)

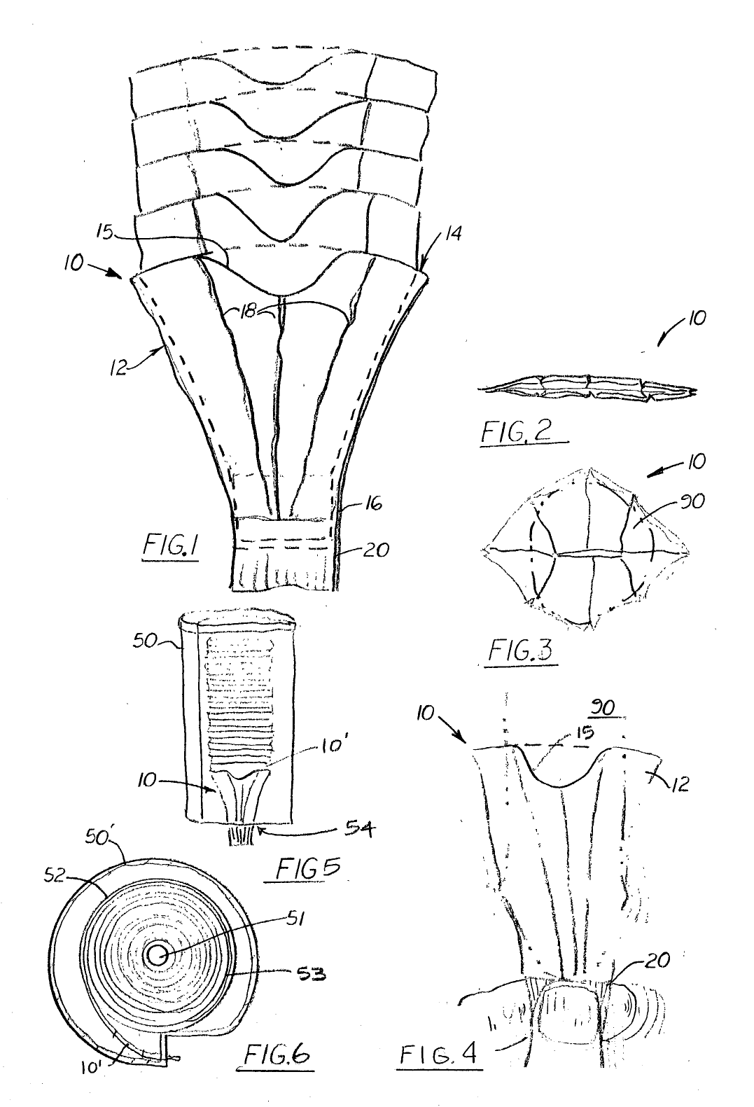

A male, post-void, urinary drip napkin 10 is shown in the accompanying FIGS. 1-6 that includes a flat, conical-shaped body 12 with a wide open end 14 and an opposite narrow closed end 16. Formed on the opposite sides of the body 12 are baffles 18 which enable the body 12 to be folded and expanded from a flat configuration shown in FIG. 2 into a conical configuration, shown in FIG. 3, capable of being aligned longitudinally over the end of a penis.

In the preferred embodiment, the body 12 is a continuous structure with no side seams that increase bulk. Formed on the closed end 16 of the body 12 is a longitudinally aligned, central tab member 20 which enables a user to easily pull the napkin 10 longitudinally from a dispenser 50, longitudinally align the napkin 10 over the penis 90, and then shake the penis, as shown in FIG. 4, with one hand.

The napkin 10 is relatively small and made of lightweight, absorbent paper material capable of being discarded in the urinal or toilet without obstructing the plumbing. In the preferred embodiment, the body 12 is made of light tissue or toilet paper and measures approximately three inches in length. The open end 14 of the body 12, which, when expanded into an open configuration, measures approximately 2½ inches in diameter. The tab member 20 is integrally formed on the body 12 and is also made of tissue or toilet paper and measures approximately ¾ inch in width and ¾ inch in length. The top edge 15 of the open end 14 is curved inward, which enables the user or his assistant to view the top surface of the penis 90 when the napkin 10 is placed thereon.

The first embodiment of the dispenser 50, shown in FIG. 5, includes a rigid, rectangular-shaped outer container 52 designed to store a plurality of longitudinally aligned napkins 10. The lower napkin 10 is positioned over the adjacent inside napkin 10′ and is interconnected so that when a lower napkin 10 is pulled from the container 52, the next napkin 10′ is pulled to automatically extend through the container's lower opening 54. The folded baffles 18 on the adjacent napkins 10′ are overlapping so that the upper napkin 10′ is partially pulled through the dispenser 50 when the lower napkin 10 is pulled.

In another embodiment, shown in FIG. 6, the dispenser 50′ is designed to hold a plurality of napkins 10 that are longitudinally aligned and connected in an end-to-end manner and collected on a roll 53, which then rotates on a central roller 51 housed inside a dispenser 50′. The adjacent napkins 10 are separated by pulling along perforated lines (not shown) formed between them.

As disclosed herein is the method for dislodging urine from a penis comprising the following steps: (a) selecting a napkin that includes a conical-shaped body with a wide open end an opposite, closed end, said body including a single tab centrally and longitudinally aligned and extending from said closed end; (b) grasping said tab and longitudinally aligning said body so that a penis may be inserted through said open end of said body; and, (c) inserting a penis into said open end; and (d) shaking said body while holding said tab to dislodge urine from the penis.

In compliance with the statute the invention, described herein has been described in language more or less specific as to structural features. It should be understood, however, the invention is not limited to the specific features shown, since the means and construction shown comprise only the preferred embodiments for putting the invention into effect. The invention is, therefore, claimed in any of its forms or modifications within the legitimate and valid scope of the amended claims, appropriately interpreted in accordance with the doctrine of equivalents.

I claim:

1. A male, post-void, urinary drip napkin, comprising:
a. a body having a wide open end and an opposite, closed end, said body capable of being stored in a flat, folded configuration and selectively expanded into a conical configuration capable of being placed around a penis, said body made of water soluble paper for disposal in a toilet; and,
b. a single tab centrally and longitudinally aligned and extending from said closed end of said body, said tab enabling said napkin to be held with one hand to longitudinally align said body over a penis and shake the penis to dislodge urine.

2. The male, post-void, urinary drip napkin, as recited in claim 1, wherein said body includes an inward curved edge located around said open end thereby enabling a penis to be viewed when extended through said wide open end and into said body.

3. The male, post-void, urinary drip napkin, as recited in claim 2, wherein said napkin is dispensed from a dispenser capable of holding a plurality of napkins aligned in a stacked configuration inside said dispenser.

4. The male, post-void, urinary drip napkin, as recited in claim 3, wherein said napkin is joined in an end-to-end manner and is dispensed from a roll containing a plurality of napkins.

5. A method for dislodging urine from a penis, comprising the following steps:
a. selecting a napkin that includes a conical-shaped body with a wide open end and an opposite, closed end, said body including a single tab centrally and longitudinally aligned and extending from said closed end;
b. grasping said tab and longitudinally aligning said body so that a penis may be inserted through the open end of said body;
c. inserting a penis into said open end; and,
d. shaking said body while holding said tab to dislodge urine from the penis.
